(19) United States
(12) Patent Application Publication
(10) Pub. No.: US 2019/0075747 A1
(43) Pub. Date: Mar. 14, 2019

(54) BICOLOR IMPATIENS VARIETIES AND METHOD OF BREEDING THE SAME

(71) Applicant: Dümmen Group B.V., De Lier (NL)

(72) Inventor: Ruth KOBAYASHI, Encinitas, CA (US)

(21) Appl. No.: 16/030,992

(22) Filed: Jul. 10, 2018

Related U.S. Application Data
(60) Provisional application No. 62/530,673, filed on Jul. 10, 2017.

(51) Int. Cl. A01H 6/16; A01H 5/02; A01H 3/04

(52) U.S. Cl. CPC A01H 6/165; A01H 3/04; A01H 5/02

(57) ABSTRACT

Provided herein are New Guinea Impatiens cultivars comprising bleeding color petals. Methods for breeding the disclosed Impatiens are also provided.

This application claims the benefit of U.S. Provisional Patent Application No. 62/530,673, filed Jul. 10, 2017, the entirety of which is incorporated herein by reference.

### BACKGROUND OF THE INVENTION

### 1. Field of the Invention

The present embodiments provided herein relate generally to horticultural, plant breeding and plant genetics.

### 2. Description of Related Art

Impatiens has become an increasingly important ornamental crop. In 1970, in order to increase the germplasm pool for this crop, the U.S. Department of Agriculture introduced 23 Impatiens from New Guinea, 1 from Celebes and 1 from Java. H. F. Winters, Am. Hotic. 52: 923 (1973). New Guinea Impatiens (NGI) encompasses a group of interbreeding species that include I. schlecteri Warb., I. herzogii K. Schum, I. linearifolia Warb., I. mooreana Schltr., I. hawkeri Bull, and other species of the same geographic origin which are interfertile. NGI are diverse phenotypically, producing large flowers with colors ranging from white to various shades of lavender, red, pink and orange. The leaves are of various shapes and sizes, with and without variegations. C Grey-Wilson, Kew Bulletin 34: 661 (1979) Although diverse phenotypically members of NGI are interfertile and generally have a 2n chromosome number of 32, T Arisumi, J. Hered., 64: 77 (1973).

NGI cultivars to date have solid color or bicolor petals. In the bicolor genotypes, the pattern of petal color has been either in a ‘star’ pattern where one color laterally bisects the petal of another color or diffuse blending of 2 colors (FIG. 1).

### SUMMARY OF THE INVENTION

In a first embodiment of the invention there is provided a New Guinea Impatiens plant comprising at least one flower with at least one petal having a bleeding color pattern phenotype, wherein the plant is obtained by introgression of the bleeding color pattern trait from a plant grown from a seed deposited at NCIMB, said plant having been selected for a bleeding color pattern phenotype. In some aspects, substantially all the flowers produced by said plant comprise the bleeding color pattern. In certain aspects, the plant may additionally comprise at least one horticultural elite trait. For example, the horticultural elite trait may be selected from short stature, upright growth, strong branches, large blooms, vigorous growth, disease resistance, insect resistance, herbicide tolerance, increased longevity, increased period of flowering, drought tolerance, uniform plant habit, a desired leaf color, a desired morphology, or flowers with desirable colors. In additional aspects, the plant comprises leaves that are medium green, dark green or variegated green. In further aspects, plants may comprise flowers with a primary color of pink, red, lavender, purple, magenta, salmon, or scarlet, and an accent color of red, purple, lavender, salmon, or orange.

In a further embodiment there is provided a progeny plant of a New Guinea Impatiens in accordance with the embodiments and aspects described herein, said progeny plant having at least one flower with at least one petal having a bleeding color pattern phenotype.

Yet a further embodiment of the invention provides a tissue culture of regenerable cells of the New Guinea Impatiens plant of any of the embodiments and aspects described herein. In certain aspects, the regenerable cells are from embryos, meristematic cells, pollen, leaves, petals, roots, root tips, anther, pistil, seed or stem. In further embodiments there is provided a plant part of the New Guinea Impatiens plant of any of the embodiments and aspects described herein. In some aspects, the part is a seed, a stalk, a petal, a bud, a leaf or a root. In additional aspects, said part can be regenerated into a plant comprising a bleeding color pattern phenotype.

In yet still a further embodiment there is provided a method of producing a New Guinea Impatiens plant comprising crossing a plant of the embodiments and aspects described herein with a second New Guinea Impatiens plant and selecting a progeny plant comprising the bleeding color pattern phenotype. In specific aspects, the second New Guinea Impatiens plant may comprise at least a first horticulturally elite trait. In further aspects, the method additionally comprises selecting a progeny plant comprising the bleeding color pattern phenotype and the first horticulturally elite trait.

Still yet a further embodiment of the invention provides a method for obtaining a New Guinea Impatiens plant according to the embodiments and aspects described herein comprising the step of introgression of the bleeding color pattern trait from a plant grown from the seed deposited at NCIMB. Further embodiments provide a New Guinea Impatiens seed wherein, upon growth, the seed produces a plant having a bleeding color pattern phenotype and comprises a trait that was introgressed from a plant grown from the seed deposited at NCIMB.

Another embodiment provides a New Guinea Impatiens plant comprising at least one flower with at least one petal having a bleeding color pattern phenotype, wherein the plant is obtained by mutation with irradiation. In some aspects, substantially all the flowers produced by said plant comprise the bleeding color pattern. In certain aspects, the plant may additionally comprise at least one horticultural elite trait. For example, the horticultural elite trait may be selected from short stature, upright growth, strong branches, large blooms, vigorous growth, disease resistance, insect resistance, herbicide tolerance, increased longevity, increased period of flowering, drought tolerance, uniform plant habit, a desired leaf color, a desired morphology, or flowers with desirable colors. In additional aspects, the plant comprises leaves that are medium green, dark green or variegated green. In further aspects, plants may comprise flowers with a primary color of pink, lavender, purple, magenta, salmon, or scarlet, and an accent color of purple, lavender, salmon, or orange.

As used herein in the specification and claims, “a” or “an” may mean one or more. As used herein in the specification and claims, when used in conjunction with the word “comprising”, the words “a” or “an” may mean one or more than one. As used herein, in the specification and claims, “another” or “a further” may mean at least a second or more.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1A. Examples of New Guinea Impatiens with solid color phenotype.

FIG. 1B. Examples of New Guinea Impatiens with ‘star’ pattern phenotype.

FIG. 1C. Examples of New Guinea Impatiens with diffuse pattern bicolor phenotype.

FIG. 2. Examples of New Guinea Impatiens with bleeding color pattern phenotype.

### DESCRIPTION OF ILLUSTRATIVE EMBODIMENTS

### I. The Present Embodiments

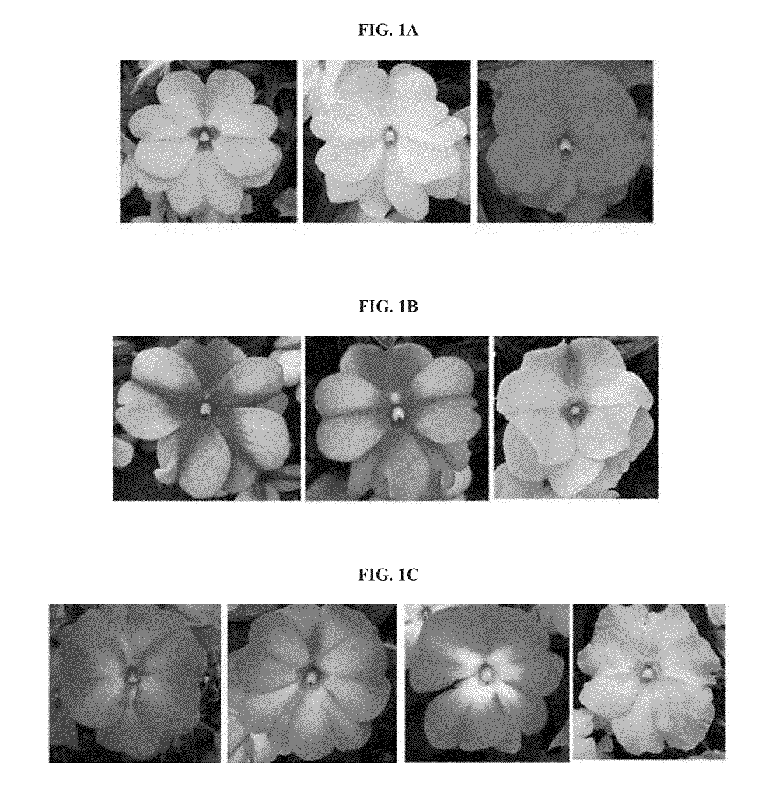

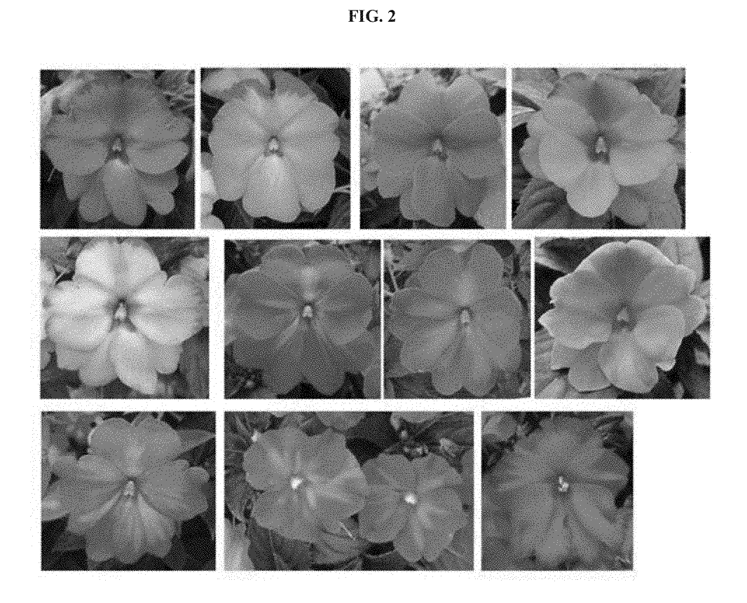

In populations of solid color New Guinea Impatiens cuttings treated with irradiation a novel bicolor petal phenotype was discovered, wherein the petals have a primary color (majority of the petal area) and an accent color either along the outer edge of a petal or radiating from the inner edge of a petal (FIG. 2). The novel bicolor petal phenotype is referred to herein as a “bleeding color pattern” or “bleeding color”. The bleeding color pattern may appear on one or more petals of a flower. The bleeding color pattern phenotype may also be acquired by intercrossing other genotypes (for example, those shown in FIGS. 1A-C) with a bleeding color genotype.

### II. Definitions

As used herein, “bleeding color” or “bleeding color pattern” are each defined as a petal pattern having a primary color (majority of the petal area) and an accent color either along the outer edge of a petal or radiating from the inner edge of a petal. A NGI plant may be described as having a bleeding color phenotype if the bleeding color pattern is observed on at least one petal of one flower.

As used herein, “single”, “single-type”, or “singleness” are each defined as the typical New Guinea Impatiens (NGI) plant which produces flowers having five petals per flower or the typical NGI flower which has five petals.

As used herein, “semi-double”, “semi-double-type”, or “semi-doubleness” are each defined as a NGI plant which produces one or more flowers having a sixth full or partial petal per flower or a NGI flower which has a sixth full or partial petal.

As used herein, “double”, “double-type” or “double-flowering” are each defined as a Impatiens plant which produces one or more flowers having at least 7 full or partial petals per flower or a Impatiens flower which has at least 7 full or partial petals. Double-flowering Impatiens cultivars are genetically stable. Double-flowering cultivars can be stably reproduced by means of asexual propagation. The characteristic of doubleness can be predictably bred into diverse single-type and semi-double-type Impatiens genetic backgrounds.

Horticultural Elite—

Plants exhibiting desired horticultural traits are considered to be horticultural elite, viz. genetic traits. Traits that may be considered to confer elitism are good longevity, large blooms and/or tolerance to pests, tolerance to disease, long flowering time, and the like.

Genetic Transformation—

A process of introducing a DNA molecules (e.g., a vector or expression cassette) into a cell or protoplast in which that exogenous DNA is incorporated into a chromosome or is capable of autonomous replication.

Introgression—

The process of transferring a genetic trait (e.g., a resistance phenotype) from one genotype to another.

### III. Deposit Information

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Breeding of NGI Plants with Bleeding Color Pattern Petals

Cuttings of solid color NGI were treated with irradiation (35 Gy of 4 MeV x-ray) from a medical linear accelerator. The treated cuttings were transplanted and grown out for approximately 6 to 8 weeks in an environment suitable to promote vegetative growth. Shoot tip cuttings from these treated plants were then harvested every 2 weeks from the treated plants for a period of 10 to 12 weeks (5 to 6 sets of cuttings). The bleeding color pattern on 1 to 3 petals were observed in some genotypes in each set of cuttings. The bleeding color pattern was found to be stable among clonally propagated selections. Phenotypes exhibiting the bleeding color pattern were propagated by vegetative cutting and maintained.

Additionally, cuttings of genotypes with the bleeding color pattern on 1 to 3 petals were harvested and treated with irradiation and cuttings were harvested in the same manner as described above. The bleeding color pattern was observed on all 5 petals on some plants.

The bleeding color phenotype can also be introgressed into any desired NGI cultivar, such as a horticulturally elite line or a line having semi-double or double flowers. Foliage and plant growth habit are similar to other standard, commercially available New Guinea Impatiens.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,399,798
U.S. Pat. No. 5,684,225
H. F. Winters, Am. Hotic. 52: 923 (1973).
C. Grey-Wilson, Kew Bulletin 34: 661 (1979)
T. Arisumi, J. Hered., 64: 77 (1973).

1. A New Guinea Impatiens plant comprising at least one flower with one petal having a bleeding color pattern phenotype wherein the plant is obtained by introgression of the bleeding color pattern trait from a plant grown from the seed deposited at NCIMB, said plant having been selected for a bleeding color pattern phenotype.

2. The New Guinea Impatiens plant according to claim 1 wherein substantially all the flowers produced by said plant comprise the bleeding color pattern phenotype.

3. The New Guinea Impatiens plant of claim 1, further comprising at least one horticultural elite trait.

4. The New Guinea Impatiens plant according to claim 3, wherein said horticultural elite trait is selected from short stature, upright growth, strong branches, large blooms, vigorous growth, disease resistance, insect resistance, herbicide tolerance, increased longevity, increased period of flowering, drought tolerance, uniform plant habit, a desired leaf color, a desired morphology, or flowers with desirable colors.

5. The New Guinea Impatiens plant of claim 1, comprising leaves that are medium green, dark green or variegated green.

6. The New Guinea Impatiens plant of claim 1, comprising flowers with a primary color of pink, lavender, purple, magenta, salmon, orange or scarlet, and an accent color of purple, lavender, salmon, or orange.

7. The New Guinea Impatiens plant of claim 1, comprising flowers with a primary color of lavender, magenta, red or orange.

8. A progeny plant of a New Guinea Impatiens in accordance with claim 1, said progeny plant having a bleeding color pattern phenotype.

9. A tissue culture of regenerable cells of the New Guinea Impatiens plant of claim 1.

10. (canceled)

11. A plant part of the New Guinea Impatiens plant of claim 1.

12-13. (canceled)

14. A method of producing New Guinea Impatiens plant comprising crossing a plant of claim 1 with a second New Guinea Impatiens plant and selecting a progeny plant comprising the bleeding color pattern phenotype.

15. The method of claim 14, wherein the second New Guinea Impatiens plant comprises at least a first horticulturally elite trait.

16. The method of claim 15, further comprising selecting a progeny plant comprising the bleeding color pattern phenotype and the first horticulturally elite trait.

17. A method for obtaining a New Guinea Impatiens plant according to claim 1 comprising the step of introgression of the bleeding color pattern trait from a plant grown from the seed deposited at NCIMB.

18. A New Guinea Impatiens seed wherein, upon growth, the seed produces a plant having a bleeding color pattern phenotype and comprises a trait that was introgressed from a plant grown from the seed deposited at NCIMB.

19. A New Guinea Impatiens plant comprising at least one flower with one petal having a bleeding color pattern phenotype wherein the plant is obtained by mutation with irradiation.

20. The New Guinea Impatiens plant according to claim 19 wherein substantially all the flowers produced by said plant comprise the bleeding color pattern phenotype.

21. The New Guinea Impatiens plant of claim 19, further comprising at least one horticultural elite trait.

22. The New Guinea Impatiens plant according to claim 21, wherein said horticultural elite trait is selected from short stature, upright growth, strong branches, large blooms, vigorous growth, disease resistance, insect resistance, herbicide tolerance, increased longevity, increased period of flowering, drought tolerance, uniform plant habit, a desired leaf color, a desired morphology, or flowers with desirable colors.

23-24. (canceled)

25. A progeny plant of a New Guinea Impatiens in accordance with claim 19, said progeny plant having a bleeding color pattern phenotype.

26-30. (canceled)
